(19) United States
(12) Patent Application Publication
(10) Pub. No.: US 2017/0311884 A1
(43) Pub. Date: Nov. 2, 2017

(54) PORTABLE ELECTRONIC APPARATUS

(71) Applicant: SEIKO EPSON CORPORATION, Tokyo (JP)

(72) Inventors: Yasutomo TAKAHASHI, Matsumoto-shi (JP); Tetsu NAKAYAMA, Matsumoto-shi (JP)

(73) Assignee: SEIKO EPSON CORPORATION, Tokyo (JP)

(21) Appl. No.: 15/484,993

(22) Filed: Apr. 11, 2017

(30) Foreign Application Priority Data
Apr. 27, 2016 (JP) 2016-088678

(51) Int. Cl. A61B 5/00; A61B 5/11; A44C 5/00

(52) U.S. Cl. CPC A61B 5/681; A44C 5/0069; A61B 5/1118

(57) ABSTRACT

A portable electronic apparatus includes a band to be mounted on a test subject. The band includes a plurality of fitting holes, a first surface as a surface facing to the test subject when mounted, and a second surface as a surface facing to an opposite side to the test subject when mounted, a contour line on the first surface side in a cross-section along a short-side direction of the band has a convex part. In the case of defining the surface corresponding to the second surface as a reference surface, and a position corresponding to the fitting hole in the first surface as a hole position, a height from the reference surface to the hole position is lower than a height from the reference surface to the convex part.

### CROSS-REFERENCES TO RELATED APPLICATIONS

This application claims priority to Japanese Patent Application No. 2016-088678, filed Apr. 27, 2016, the entirety of which is herein incorporated by reference.

### BACKGROUND

### 1. Technical Field

The present invention relates to a portable electronic apparatus and so on.

### 2. Related Art

In recent years, necessity of sensing physical activity information has been increasing not only in a conventional physical activity meter but also in sport equipment used when performing a strenuous movement such as a running watch. Further, there has been invented a band used for fixing such a physical activity meter, a watch (timepiece), or the like to, for example, an arm of the test subject. As related art of such a technology, there can be cited technologies disclosed in, for example, JP-A-2010-110634 (Document 1) and JP-A-07-213312 (Document 2).

In the sport equipment used when performing the strenuous movement such as a running watch described above, a region to which a band is mounted becomes stuffy to cause an itch, a pain, a rash, or the like in some cases. Therefore, it is necessary to improve the airflow in the mounting region of the band to make it difficult to become stuffy.

As a general method of improving the airflow in the mounting region of the band, there can be cited a method of providing a number of through holes to the band to expose a part of the skin through the band to have contact with air. According to this method, since the ridge lines formed of the through holes and the reverse surface of the band have contact with the skin, in the case of tightening the band strongly, the shapes of the holes remain on the skin as a trace in some cases. Moreover, since an itch or a pain occurs along the lines of the hole shapes in some cases, it is insufficient only to provide the holes as a measure against the problem described above.

Further, if providing the number of holes, the appearance is also affected to degrade the dignity in some cases. Although there is no problem in sport watches worn in an exercise, the appearance is also important with respect to the equipment such a physical activity meter used in a scene of a social activity such as a conference or a business meeting, and also in every day life.

Regarding these points, in Document 1, there is no description mentioning the wearing feeling of a wristwatch-type biological information measurement device. Further, in Document 2, although a variety of configurations are disclosed with respect to the shape of the watchband, there is no mention of the problems inherent in the biological information measurement device such as fixing the device firmly to the wrist so as to prevent the case section from being displaced or continuing to wear the device for a long period of time.

### SUMMARY

An advantage of some aspects of the invention is to provide a portable electronic apparatus or the like capable of suppressing the discomfort while wearing the band.

An aspect of the invention relates to a portable electronic apparatus including a band adapted to be mounted on a test subject, wherein the band includes a plurality of fitting holes, a first surface as a surface facing to the test subject when mounted, and a second surface as a surface facing to an opposite side to the test subject when mounted, a contour line on the first surface side in a cross-section along a short-side direction of the band has a convex part, and in a case of defining a surface corresponding to the second surface as a reference surface, and a position corresponding to the fitting hole in the first surface as a hole position, a height from the reference surface to the hole position is lower than a height from the reference surface to the convex part.

In the aspect of the invention, the contour line on the first surface side in the cross-section along the short-side direction of the band has the convex part, and the height from the reference surface corresponding to the second surface of the band to the hole position on the first surface side is lower than the height from the reference surface to the convex part. Therefore, the region corresponding to the convex part in the first surface abuts on the mounting region of the test subject to form a region not abutting on the test subject in the first surface to thereby improve the airflow when mounting the band. Therefore, it becomes possible to prevent the discomfort caused while wearing the band.

In the aspect of the invention, the contour line on the first surface side in the cross-section along the short-side direction of the band may have the convex part and a concave part.

With this configuration, it becomes possible to form a space between the region corresponding to the concave part of the contour line on the first surface and the mounting region of the test subject.

In the aspect of the invention, in a case of defining the height from the reference surface to the hole position as h1, the height from the reference surface to the convex part as h2, and a height from the reference surface to the concave part as h3, h2>h1>h3 may be true.

With this configuration, it becomes possible to form a region protruding to a level higher than the fitting hole with respect to the reference surface and a region recessed to a level lower than the fitting hole with respect to the reference surface in the cross-section along the short-side direction of the band.

In the aspect of the invention, the band may have a first side surface and a second side surface each crossing the first surface and the second surface, and in a case of defining a position corresponding to the first side surface and the second side surface in the first surface as a side surface position, a height from the reference surface to the convex part as h2, and a height from the reference surface to the side surface position as h4, h2>h4 may be true.

With this configuration, it is possible to prevent the contour line on the first surface side in the side surface of the band from abutting on the mounting region of the test subject when mounting the band to make the test subject feel the pain or to make the trace of the band remain on the skin.

In the aspect of the invention, in a case of defining a height from the reference surface to the concave part as h3, h2>h4>h3 may be true.

With this configuration, it becomes possible to make the height of the side surface of the band from the reference surface constant in, for example, the cross-section in the short-side direction of the band.

In the aspect of the invention, 0.10 mm≦(h2−h1)≦1.0 mm may be true.

With this configuration, it becomes possible to form an entrance path of the air between the band and the skin of the test subject while ensuring the thinness of the band.

In the aspect of the invention, the fitting hole may be formed at a position except the highest position of the convex part.

With this configuration, it becomes possible to prevent the edge of the fitting hole from abutting on the mounting region of the test subject while, for example, mounting the band to cause the test subject to feel the pain, or to provide a trace of the fitting hole to the mounting region.

In the aspect of the invention, the contour line on the first surface side in the cross-section along the short-side direction of the band may have the convex part and a concave part, and the fitting hole may be formed at a position except the highest position of the convex part and the lowest position of the concave part.

With this configuration, it becomes possible to prevent the edge of the fitting hole and the contour line on the first surface side of the side surface of the band from abutting on the mounting region of the test subject while, for example, mounting the band to cause the test subject to feel the pain, or to provide a trace of the fitting hole to the mounting region.

In the aspect of the invention, the contour line of the fitting hole on the first surface side in the cross-section along the short-side direction of the band maybe tilted with respect to the reference surface.

With this configuration, it becomes possible to make the height from the reference surface to one end of the fitting hole and the height from the reference surface to the other end of the fitting hole different from each other.

In the aspect of the invention, the contour line on the first surface side in the cross-section along the short-side direction of the band may be point symmetrical about the hole position.

With this configuration, it becomes possible to set, for example, the shape of the contour line on the first surface in the cross-section in the short-side direction of the band to the reversed shape cantered on the hole position.

In the aspect of the invention, in the band, a contour line on the first surface side in a cross-section along a long-side direction of the band may have a wavy shape.

With this configuration, it becomes possible, for example, to form the wavy shape in both of the contour line in the short-side direction and the contour line in the long-side direction in the first surface of the band.

In the aspect of the invention, the contour line on the first surface side in the cross-section along the short-side direction of the band may have a convex part and a flat part, and the fitting hole is disposed in the flat part.

With this configuration, it becomes possible, for example, to make it easy for the air to enter the region corresponding to the flat part of the first surface of the band.

In the aspect of the invention, the portable electronic apparatus may further include a physical activity information detection section adapted to detect physical activity information of the test subject.

With this configuration, it becomes possible, for example, to obtain the physical activity information of the teat subject while wearing the portable electronic apparatus.

Another aspect of the invention relates to a portable electronic apparatus including a band adapted to be mounted on a test subject, wherein the band includes a plurality of fitting holes, a first surface as a surface facing to the test subject when mounted, and a second surface as a surface facing to an opposite side to the test subject when mounted, a contour line on the first surface side in a cross-section along a short-side direction of the band has a convex part, and in a case of defining a surface corresponding to the second surface as a reference surface, the fitting hole is formed at a position except a position where a distance from the reference surface becomes the longest in the cross-section along the short-side direction of the band.

### BRIEF DESCRIPTION OF THE DRAWINGS

The invention will be described with reference to the accompanying drawings, wherein like numbers reference like elements.

FIG. 1 is a perspective view of an upper surface (an obverse surface) of a portable electronic apparatus according to an embodiment of the invention.

FIG. 2 is a perspective view of a lower surface (a reverse surface) of the portable electronic apparatus according to the embodiment.

FIG. 3 is an explanatory diagram of a mounting state of the portable electronic apparatus.

FIG. 4 is a perspective view of a reverse surface of a band.

FIG. 5 is a perspective view of an obverse surface of the band.

FIG. 6 is an appearance diagram of the reverse surface of the band.

FIG. 7 is an appearance diagram of the obverse surface of the band.

FIG. 8 is an appearance diagram of a side surface of the band.

FIG. 9 is another appearance diagram of the side surface of the band.

FIG. 10 is another appearance diagram of the side surface of the band.

FIG. 11 is another appearance diagram of the side surface of the band.

FIG. 12 is a cross-sectional view along the long-side direction of the band.

FIG. 13 is another cross-sectional view along the long-side direction of the band.

FIG. 14 is another cross-sectional view along the long-side direction of the band.

FIG. 15 is a plan view of the side surface of the band.

FIG. 16 is a cross-sectional view along the short-side direction of the band.

FIG. 17 is another cross-sectional view along the short-side direction of the band.

FIG. 18 is a cross-sectional view along the short-side direction of a band with an uneven surface.

FIG. 19 is another cross-sectional view along the short-side direction of the band with the uneven surface.

FIG. 20 is an explanatory diagram of a contact state between the band and the mounting region of a test subject while wearing the band.

FIG. 21 is a cross-sectional view along the short-side direction of a band having a fitting hole formed in an area including a local maximum point.

FIG. 22 is a cross-sectional view along the short-side direction of a band having the fitting hole formed in an area including a local minimum point.

FIG. 23 is a perspective view of a band according to a comparative example.

FIG. 24 is a cross-sectional view along the short-side direction of a band according to a modified example.

FIG. 25 is an explanatory diagram of a portable electronic apparatus having a single band.

### DESCRIPTION OF AN EXEMPLARY EMBODIMENT

Hereinafter, an embodiment of the invention will be described. It should be noted that the embodiment described below do not unreasonably limit the contents of the invention as set forth in the appended claims. Further, all of the constituents described in the embodiment are not necessarily essential elements of the invention.

### 1. OUTLINE

In the sport equipment such as a running watch, the mounting region of the band becomes stuffy due to sweating to cause an itch, a pain, a rash, or the like in some cases. Further, a wrist device with a physical activity measurement function and a wrist device with a pulse wave measurement function are generally worn for a long period of time, and are also worn during sleep in some cases. In this case, since the band always adheres to the skin, the problem of an itch of a rash is apt to occur in the region where the band and the skin have contact with each other. Further, in the case of the wrist device with the pulse wave measurement function, since it is necessary to be wound around the wrist more tightly than an ordinary watch in order to secure sufficient accuracy for the pulse wave measurement, the adhesion to the skin becomes stronger, and a pain or the like occurs in some cases.

Therefore, to begin with, in order to prevent the stuffy state caused by sweating, it is necessary to improve the airflow in the mounting region of the band. As a general method thereof, there can be cited a method of providing a number of through holes to the band to expose a part of the skin through the band to have contact with air. According to this method, since the ridge lines formed of the through holes and the reverse surface of the band have contact with the skin, in the case of tightening the band strongly, the shapes of the holes remain on the skin as a trace in some cases. Moreover, an itch or a pain occurs along the lines of the hole shapes in some cases. Therefore, it can be said that it is insufficient only to provide the holes as the measure against the itch, the pain, and so on caused during wearing described above.

Further, if providing the number of holes, the appearance is also affected to degrade the dignity of the appearance. Therefore, there arises another problem that in the case of taking business manners into consideration, it is hard to wear the wrist device in a scene of a social activity such as a conference or a business meeting. In particular, the physical activity meter needs to be worn for a long period of time in many cases, and is desirably provided with an appearance applicable to all situations.

Therefore, in order to solve such a problem, in the present embodiment, the shape of the surface having contact with the wrist is devised in a band of a portable electronic apparatus. Specifically, in the present embodiment, the surface on the wrist side of the band of the portable electronic apparatus is made to be shaped like a continuous wave.

Thus, the contact area between the band and the wrist is reduced to make the air easy to flow between the band and the wrist. As a result, the stuffy state and wetting of the band due to sweat can be reduced. Further, since the wavy shape continues without a break, no angular region abuts on the skin, and it is hard to cause the pain or the itch. As described above, according to the present embodiment, it is possible to prevent the itch, the pain, and the rash in the mounting region of the band from occurring. Further, since no through holes for ensuring the airflow are provided to the band, the appearance is hardly affected, and it becomes possible to wear the device in all situations.

### 2. CONFIGURATION EXAMPLE OF PORTABLE ELECTRONIC APPARATUS

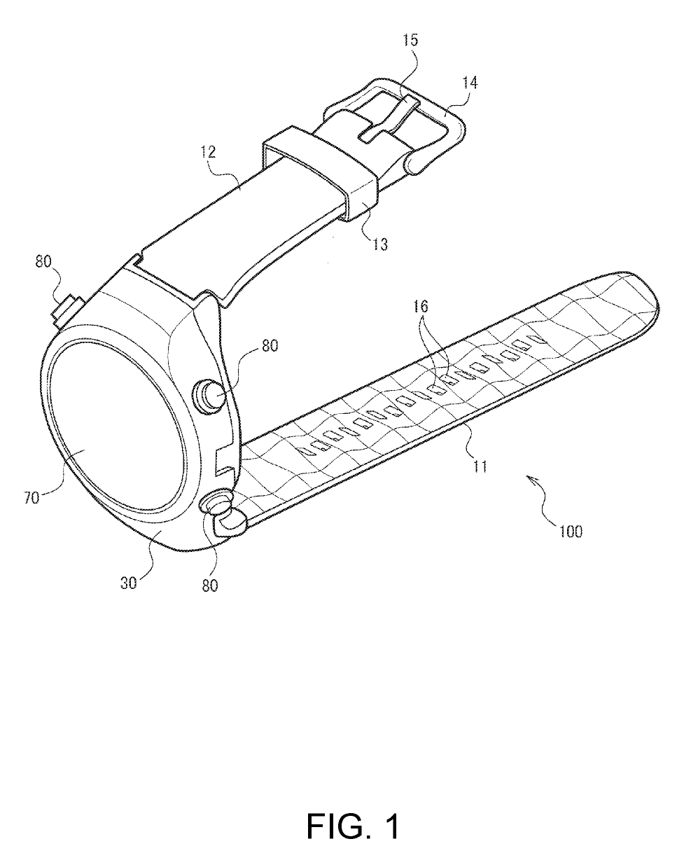

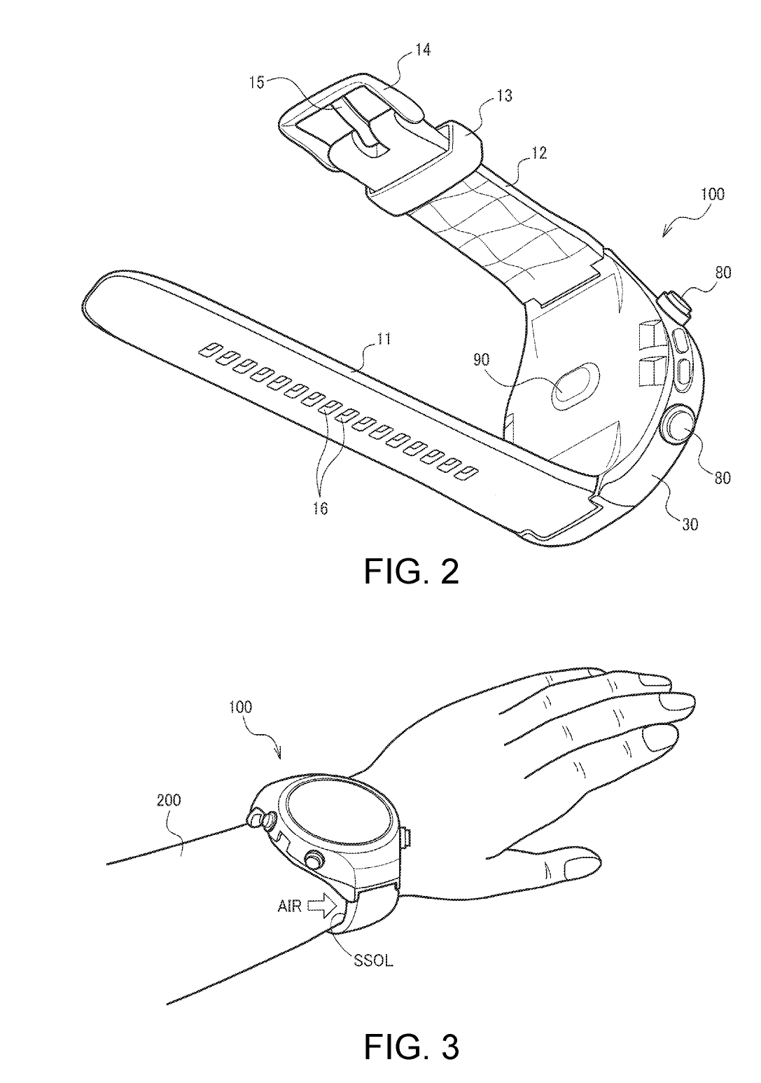

Then, a configuration example of the portable electronic apparatus 100 according to the present embodiment is shown in FIG. 1 and FIG. 2. FIG. 1 shows a perspective view of an upper surface (an obverse surface) of the portable electronic apparatus 100, and FIG. 2 shows a perspective view of a lower surface (a reverse surface) of the portable electronic apparatus 100. Further, FIG. 3 shows the state in which the portable electronic apparatus 100 shown in FIG. 1 and FIG. 2 is mounted on the wrist 200 of the test subject. It should be noted that although in FIG. 3, the portable electronic apparatus 100 is mounted on the wrist 200 of the test subject, the mounting position of the portable electronic apparatus 100 is not limited to the wrist.

The portable electronic apparatus 100 according to the present embodiment includes a first band 11, a second band 12, a case section 30, a display section 70, and an operation section 80. Further, as shown in FIG. 2, the portable electronic apparatus 100 can also be a physical activity information detection device including a physical information detection section (a sensor section) 90. It should be noted that the portable electronic apparatus 100 is not limited to the configurations shown in FIG. 1 and FIG. 2, but a variety of practical modifications such as omission of some of these constituents or addition of other constituents can be adopted. Further, here, in order to describe features of the present embodiment easy to understand, there is illustrated a wristwatch-type device with the first band 11 and the second band 12 fixed to the case section 30. However, it is also possible to adopt a configuration in which first band and the second band are integrated with each other (a band 10 shown in FIG. 25) , and the case section 30 is inserted in a housing hole disposed in between as shown in FIG. 25 described later.

Then, each section of the portable electronic apparatus 100 will be described. The first band 11 is attached to one end of the case section 30, and is provided with a plurality of fitting holes 16. The second band 12 is attached to the other end of the case section 30, and is provided with a fixation section (a free loop, a fixed loop) 13 and a buckle section (a connection section) 14. Further, the bands 11 (12) each have a first surface to be located on the test subject side when mounting the band 11 (12). Further, in the present embodiment, in the cross-sectional surface along the long-side direction of the band 11 (12), the profile line on the first surface side has the wavy shape.

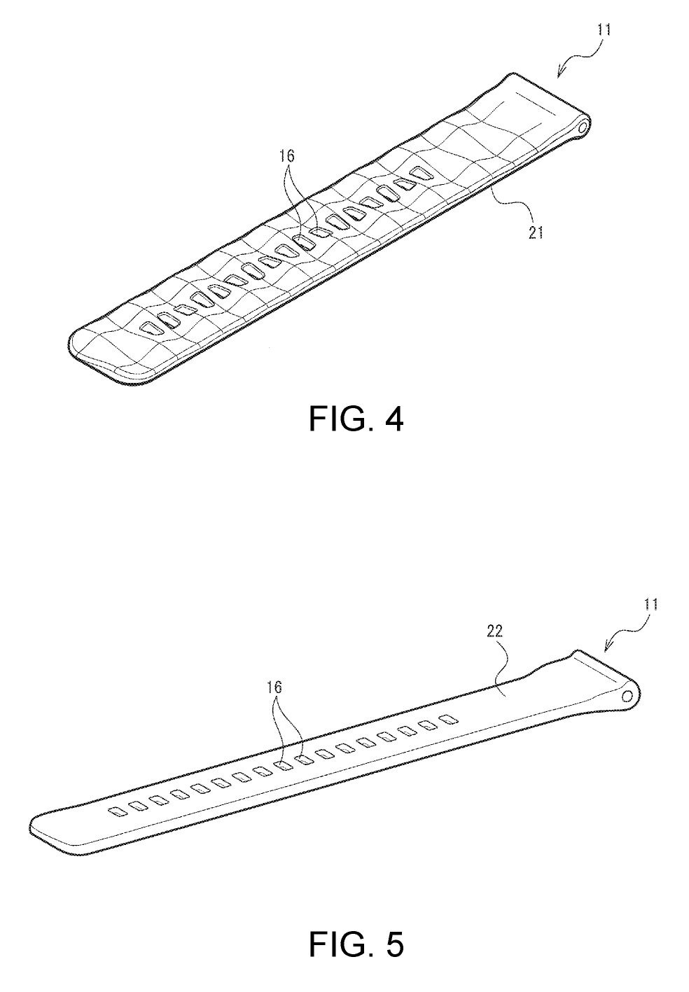

For example, FIG. 4 and FIG. 5 each show an appearance diagram (a perspective view) of the first band 11. As shown in FIG. 4 and FIG. 5, the first band 11 has a first surface 21, which is a surface facing to the test subject or a surface having contact with the test subject when mounting the first band 11 (the physical activity information detection section 90), and a second surface 22, which is a surface facing to an opposite side to the test subject or a surface not having contact with the test subject (a surface located outside) when mounting the first band 11 . It should be noted that hereinafter the first surface 21 is referred to as a reverse surface, and the second surface is referred to as an obverse surface.

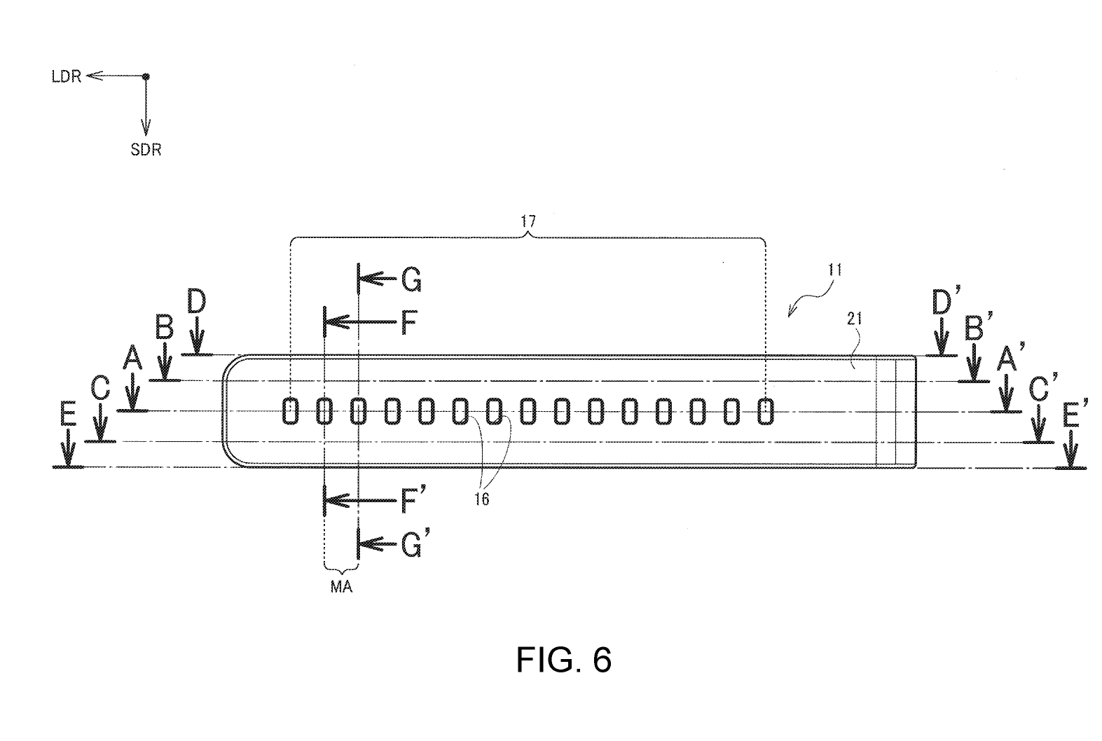

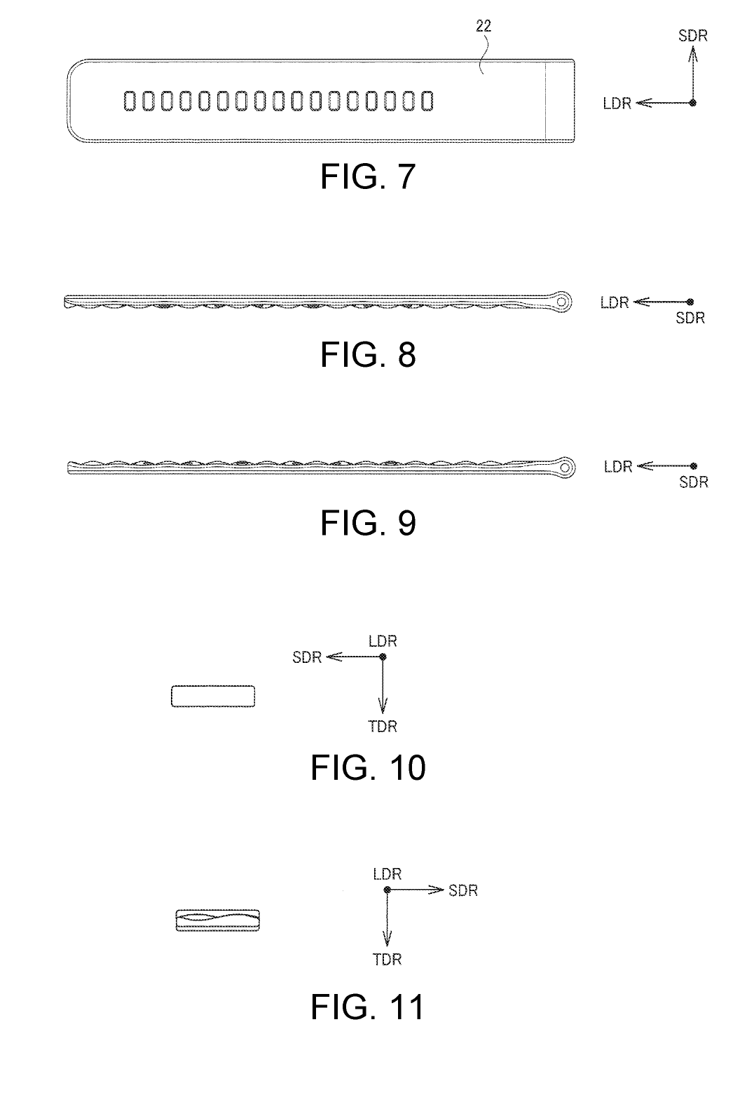

Then, FIG. 6 through FIG. 11 show six-sided view of the first band 11. FIG. 6 shows the reverse surface 21 side of the band 21, and FIG. 7 shows the obverse surface 22 side of the band 11. Further, FIG. 8 and FIG. 9 each show a side surface of the band 11, FIG. 10 shows the band 11 viewed from the side of one end to be mounted on the case, and FIG. 11 shows the band 11 viewed from the side of the other end opposite to the one end to be mounted on the case.

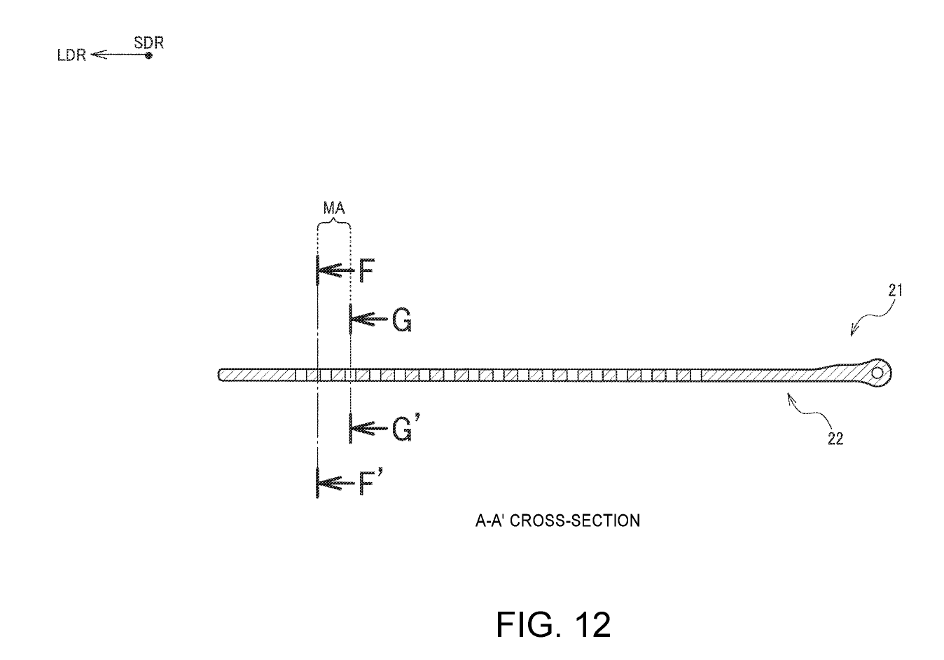

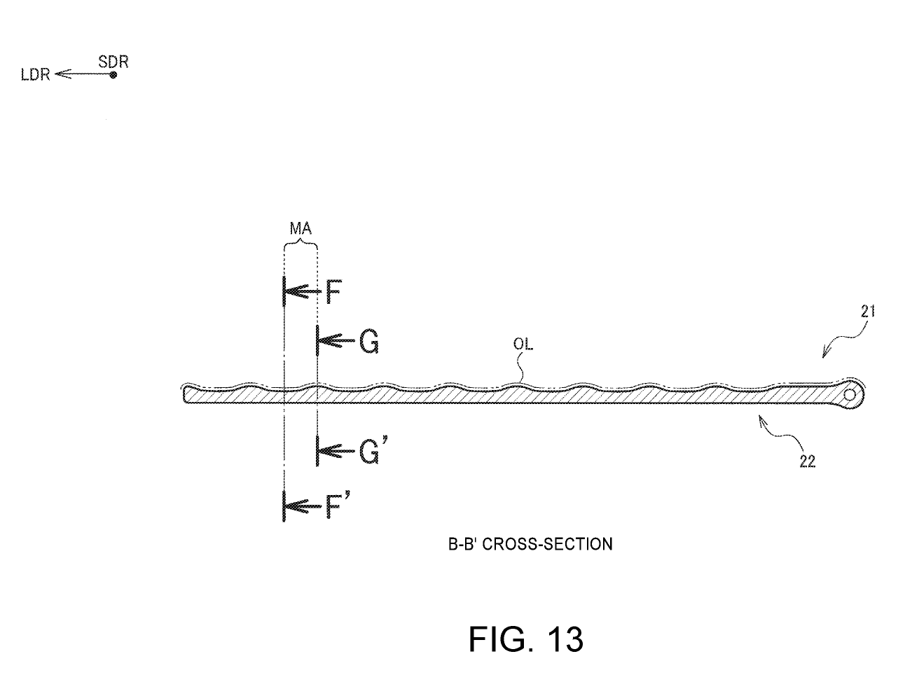

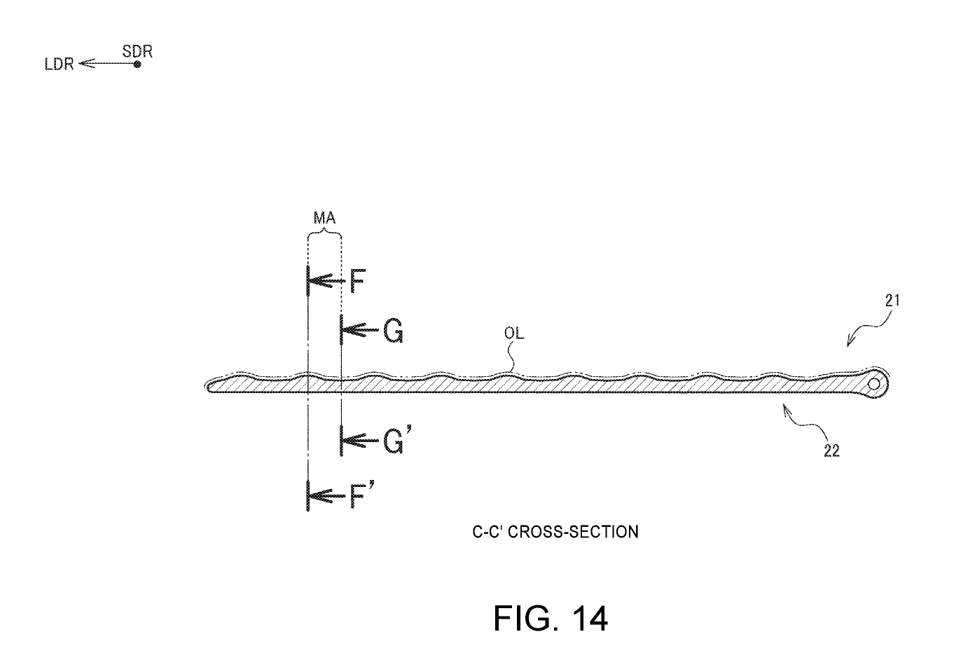

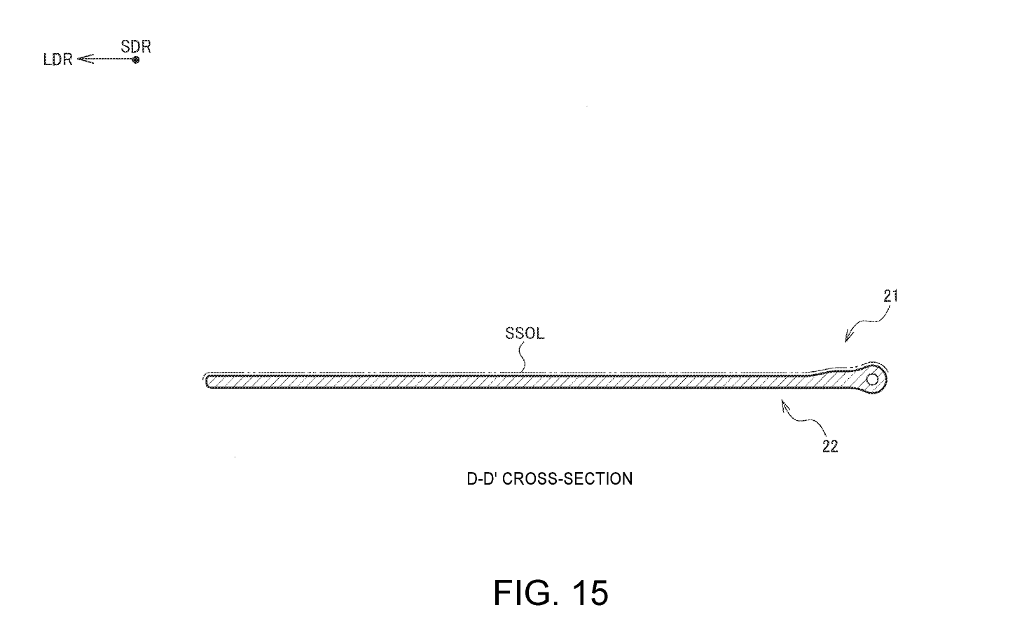

Further, FIG. 12 through FIG. 14 each show a cross-sectional view of the first band 11. FIG. 12 illustrates a cross-section in the line A-A′ along the long-side direction LDR of the band 11 shown in FIG. 6. Here, the long-side direction is the extending direction of the band 11, namely a direction from one end of the band 11 connected to the case section 30 toward the other end, or the opposite direction thereto. In the example shown in FIG. 6, the direction LDR corresponds to the long-side direction. Similarly, FIG. 13 illustrates the cross-section in the line B-B′ shown in FIG. 6, and FIG. 14 illustrates the cross-section in the line C-C′ shown in FIG. 6. Further, FIG. 15 illustrates the shape along the line D-D′ or the line E-E′ shown in FIG. 6 corresponding to the side surface of the band 11. Further, as shown in the cross-sectional views of FIG. 13 and FIG. 14, in the cross-section along the long-side direction LDR of the first band 11, the contour line on the reverse surface 21 side has a wavy shape, and the contour line on the obverse surface 22 side has a flat shape. In contrast, as shown in FIG. 12 and FIG. 15, in the positions of the line A-A′, the line D-D′, and the line E-E′ shown in FIG. 6, the contour line on the reverse surface 21 side does not have a wavy shape. It should be noted that although the shape of the first band 11 is described here, substantially the same shape can also be applied to the second band 12.

Further, the contour line (ridge line) in the cross-section denotes a line tracing the contour (outer frame, outer peripheral part) of the cross-section in the case of making a planar view of the cross-section. It should be noted that the contour of the shape located behind the cross-section is not the contour line of the cross-section. As an example, in FIG. 13, the contour line on the reverse surface 21 side in the cross-section of the band 11 denotes the line represented by a bold line OL, and the contour line OL has a wavy shape. Alternatively, the contour line (ridge line) in the cross-section can also be defined as a contour line of the cross-section in the case of making a projection view of the cross-section.

Here, as described above, if a part having contact with the skin of the wearer has a stepped part, a sharp part, or the like in the band 11 (12), a strong stimulus to the skin occurs to cause an itch or a rash.

In contrast, the shape of the reverse surface 21 of the band 11 (12) according to the present embodiment has a wavy shape continuing along the long-side direction (longitudinal direction) of the band 11 (12), and does not include the stepped part, the sharp part, and so on. Further, as described later, the wavy shape of the reverse surface 21 also continues in the short-side direction perpendicular to the long-side direction of the band 11 (12).

Here, the short-side direction denotes a direction perpendicular to (crossing) the long-side direction described above, or the opposite direction thereto. In the example shown in FIG. 6, the direction SDR corresponds to the short-side direction.

Further, the wavy shape denotes, for example, a shape formed of a plurality of unit wave shapes (unit waves) connected to each other in a cross-sectional view along the extending direction (the long-side direction) of the band 11 (12), or in a side view perpendicular to the extending direction (the long-side direction) of the band 11 (12). Further, each of the wave shapes constituting the wavy shape is, for example, a shape changing in a direction from the reverse surface 21 toward the obverse surface 22 in a range of the phase of α° to β° (α<β), and changing in a direction from the obverse surface 22 toward the reverse surface 21 in a range of the phase of β° to γ° (β<γ). As an example, α=0°, β=180°, and γ=360° can be cited. It should be noted that the wave shapes can also be different from each other. Further, modified implementation of using a half-wave shape or the like as each of the wave shapes is also possible. Further, the wavy shape can also be a shape having a contact region between the band 11 (12) and the skin, and a noncontact region repeating periodically or in an aperiodic manner. Alternatively, the wavy shape can also be a shape in which the distance from the obverse surface 22 to the reverse surface 21 of the band 11 (12) continuously changes periodically or in an aperiodic manner in a cross-sectional view along the extending direction (the long-side direction) of the band 11 (12). Further, the wavy shape can also be a shape having a point at which the distance from the obverse surface 22 to the reverse surface 21 of the band 11 (12) has a local maximum, and a point at which the distance has a local minimum. Such a wavy shape is the shape having an effect of reducing the contact area between the skin and the band 11 (12) , and at the same time the shape weak in stimulus to the skin. Therefore, a pain is hard to be caused, and a trace is hard to remain on the skin after used. Since it is particularly easy for a person soft in skin such as a person rich in subcutaneous fat, woman, or youth to have a trace on the skin, the effect of making it difficult to have a trace is significant.

Further, as shown in FIG. 3, since the air AIR gets through the gap of the wavy shape, the airflow is also improved, and it is possible to prevent the sweat from standing between the band 11 (12) and the mounting region such as an arm.

Therefore, according to the present embodiment, it becomes possible to prevent the itch, the pain, and the rash in the mounting region of the band 11 (12) from occurring.

Further, as described above, the obverse surface on the outer side of the band 11 (12) is not required to have the wavy shape, and can also be provided with, for example, a flat shape. Further, there is no need to provide a number of through holes in order to ensure the airflow. Therefore, it is also possible to provide an appearance, which does not damage the dignity of the appearance, and is applicable to all situations.

Then, the description of other sections will be resumed. As shown in FIG. 1 through FIG. 3, the fixation section (the free loop, the fixed loop) 13 fixes the first band 11 along the second band 12 in the state in which the first band 11 and the second band 12 are connected to each other. As shown in FIG. 1 and FIG. 2, the buckle section (a clasp section, a connecting section) 14 has a locking section 15, and connects the first band 11 and the second band 12 to each other. It is also possible for the second band 12 to use a buckle instead of the buckle section 14.

Further, the locking section 15 fits in either one of the fitting holes 16 when mounting the bands. As shown in, for example, FIG. 1, the locking section 15 is formed of a metal bar or a plastic bar.

Further, as shown in FIG. 1, the fitting holes 16 are each a hole penetrating the first band 11 in the thickness direction, and are provided to the first band 11 with regular intervals. Further, when mounting the portable electronic apparatus 100, the locking section 15 is inserted in either one of the fitting holes 16 to thereby lock the second band 12 to the first band 11. Further, by selecting the fitting hole to which the locking section 15 is inserted out of the fitting holes 16, the length of the band for clamping the mounting region of the test subject can be adjusted. It should be noted that the locking section 15 is not necessarily a single bar as shown in FIG. 1. It is also possible to adopt a configuration in which, for example, the locking section 15 has a plurality of bars, and the second band 12 is locked to the first band 11 by inserting the plurality of bars respectively in different ones of the fitting holes when mounting the portable electronic apparatus 100. Further, it is also possible for the locking section 15 to be a pinch section or the like for pinching (or nipping) to lock the first band 11. Further, the buckle section 14 and the locking section 15 are formed of a material higher in hardness than the members constituting the band such as a metal member or a resin member, but it is also possible to adopt a configuration of covering the buckle section 14 with the band member. By adopting such a configuration, the sense of unity between the buckle section 14 and the band is improved to thereby improve the appearance, and at the same time, the impact caused when having contact with the body of the user or a peripheral substance can be absorbed.

Further, as shown in FIG. 2, the case section 30 is provided with the physical activity information detection section (the sensor section) 90 for detecting the physical activity information of the test subject.

The physical information detection section 90 has a light emitting section not shown, a light receiving section not shown, and a detection window, and detects the physical activity information of the test subject in the state of being pressed against the test subject. Specifically, the test subject is irradiated with the light emitted from the light emitting section via the detection window, and then the light reflected by the test subject enters the light receiving section again via the detection window. Then, the physical activity information detection section 90 detects the physical activity information of the test subject based on the reflected light received by the light receiving section. Here, the detection window is formed of a transparent material or a translucent material for transmitting the light emitted from the light emitting section and the reflected light reflected by the test subject. More specifically, for example, the detection window is made of transparent plastic. Then, the detection window plays a role of physically separating the light emitting section and the light receiving section from the test subject while mounting the portable electronic apparatus 100. Further, the light receiving section can be realized by a light receiving element such as a photodiode, and the light emitting section can be realized by a light emitting element such as an LED.

Further, for example, the physical activity information detection section 90 is a body motion sensor, and the physical activity information is body motion information. The body motion information is information representing the body motion of the test subject obtained from the body motion sensor (the physical activity information detection section 90). The body motion information is information representing, for example, a moving distance of the test subject, the number of steps, a stride length, moving time, moving speed, acceleration, an absolute amount of an acceleration variation, a frequency of the acceleration variation, an amount exercise, a content of the exercise (a content of the physical activity), an elevation difference per certain unit time, an elevation, information obtained from a gyro sensor, an absolute amount of an angular velocity variation, a frequency of the angular velocity variation, information obtained from a geomagnetic sensor, an absolute amount of a geomagnetic variation, a frequency of the geomagnetic variation, and information obtained from an atmospheric pressure sensor signal.

As the body motion sensor (the physical activity information detection section 90), there can be used, for example, an acceleration sensor. Further, besides the acceleration sensor and so on, the body motion sensor can also be a gyro sensor, an altitude sensor, a geomagnetic sensor, an atmospheric pressure sensor, and so on. Further, the body motion sensor 300 can also be, for example, a GPS (global positioning system) receiver.

Further, the display section 70 displays a variety of types of information. The function of the display section 70 can be realized by, for example, a liquid crystal display, an organic EL display, or an electronic paper.

Further, the operation section 80 receives an operation by the user. The operation section 80 can be realized by, for example, a button, a switch, or a touch panel.

### 3. DETAILED DESCRIPTION OF BAND SHAPE

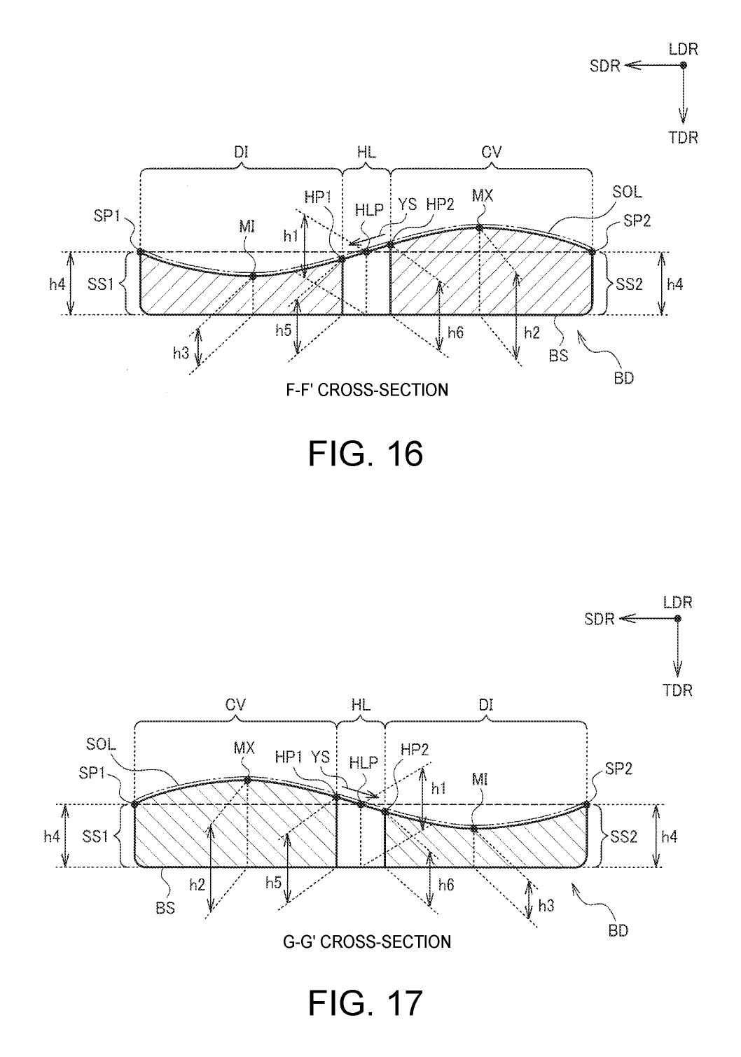

Then, the shape of the band according to the present embodiment will be described in detail using FIG. 16 through FIG. 23. In particular, the shape of the band according to the present embodiment will hereinafter be described focusing attention on the cross-section in the short-side direction of the band. For example, FIG. 16 illustrates the F-F′ cross-section of the band 11 shown in FIG. 6, and FIG. 17 illustrates the G-G′ cross-section of the band 11 shown in FIG. 6.

For example, in the example shown in FIG. 16, the contour line on the reverse surface (21 in FIG. 6) side in the cross-section (the F-F′ cross-section in FIG. 6) along the short-side direction SDR of the band BD (11 in FIG. 6) is defined as SOL. The contour line SOL denotes aline tracing the contour (outer frame, outer peripheral part) of the cross-section on the reverse surface side in the case of making the planar view of the cross-section along the short-side direction SDR as shown in FIG. 16.

Further, in the example shown in FIG. 16, a surface corresponding to the obverse surface (22 in FIG. 7) is defined as a reference surface BS, and a position corresponding to the fitting hole HL (16 in FIG. 6) in the reverse surface is defined as a hole position HLP.

In this case, the contour line SOL has a convex part CV. Further, as shown in FIG. 16, in the case in which the height from the reference surface BS to the hole position HLP is defined as h1, and the height from the reference surface BS to the convex part CV is defined as h2, the height h1 from the reference surface BS to the hole position HLP is lower than the height h2 from the reference surface BS to the convex part CV. In other words, h2>h1 is true, and thus the convex part CV is formed at a higher position than the hole position HLP.

Here, the convex part CV corresponds to a given range including a peak part MX at which the distance from the reference surface BS becomes the longest in the contour line SOL on the reverse surface side of the cross-section along the short-side direction SDR of the band BD. Further, the peak part MX denotes a given range including a peak at which the wavy shape formed by the contour line SOL is switched from rising to falling. The peak part MX can be said to be a local maximum point of the wavy shape. It should be noted that the peak part MX can also be a flat peak. For example, although in the example shown in FIG. 16 and FIG. 17, the contour line

SOL can be divided into two ranges with reference to the fitting hole HL (or the hole position HLP) , the convex part CV is the range including the local maximum point MX out of the two ranges.

Then, the hole position HLP denotes the position corresponding to the fitting hole HL in the contour line SOL on the reverse surface side of the cross-section along the short-side direction SDR of the band BD. For example, in the example shown in FIG. 16 and FIG. 17, the hole position HLP is a position corresponding to the center of the range from one end to the other end of the fitting hole HL on the contour line SOL. It should be noted that the hole position HLP is not limited thereto, but can also be defined as, for example, the position HP1 of the one end of the fitting hole HL on the contour line SOL or the position HP2 of the other end thereof.

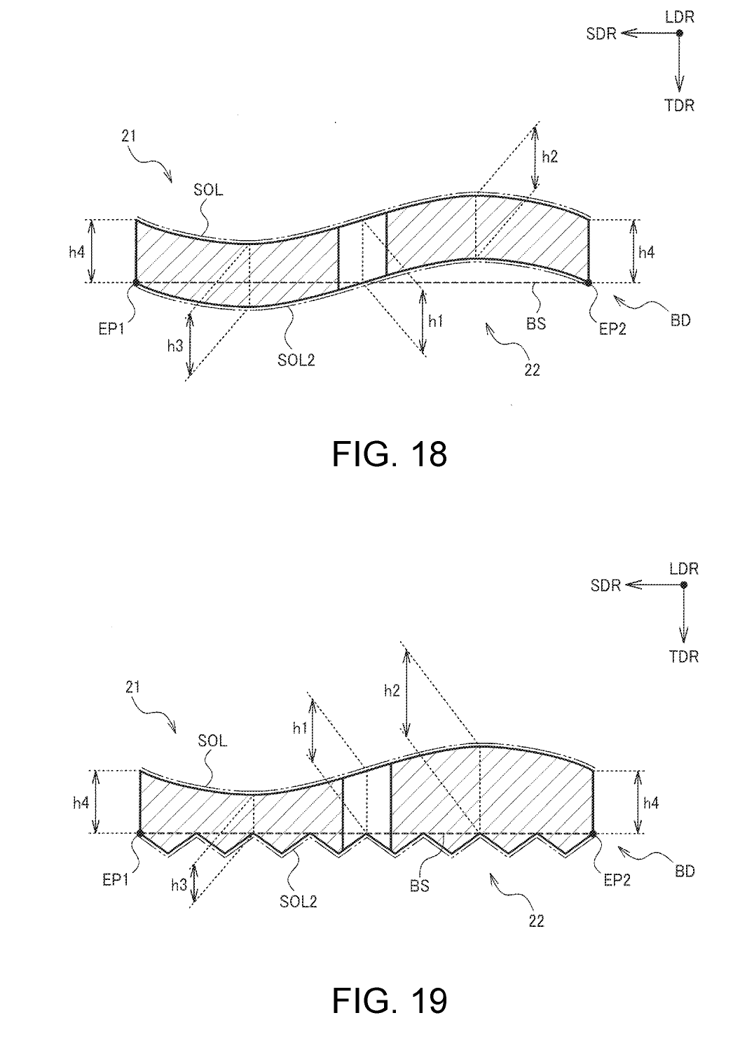

Further, the reference surface BS is an imaginary plane corresponding to the obverse surface of the band BD. For example, in the example shown in FIG. 16 and FIG. 17, the obverse surface is actually a flat surface, and the reference surface BS coincides with the obverse surface. It should noted that the present embodiment is not limited to this example, but even in the case in which the obverse surface is not a flat surface, the reference surface BS as the imaginary plane can be defined. For example, FIG. 18 and FIG. 19 each show an example of the case in which the obverse surface is not a flat surface. Similarly to FIG. 16 and FIG. 17, FIG. 18 and FIG. 19 each illustrate also the cross-section along the short-side direction SDR of the band BD. Further, in the example shown in FIG. 18, the contour line S0L2 of the obverse surface 22 has a wavy shape similarly to the reverse surface 21, and in the example shown in FIG. 19, the contour line S0L2 of the obverse surface 22 has a jagged shape (a shape including concavoconvex smaller in pitch than the wavy shape of the reverse surface 21) drawing a triangular wave. In this case, in the example shown in FIG. 18, it is possible to set an imaginary plane connecting one end EP1 of the obverse surface 22 and the other end EP2 thereof as the reference surface BS, and define the convex part CV using the height h2 from the reference surface BS. The same applies to the example shown in FIG. 19. The obverse surface 22 having such a shape as shown in FIG. 19 is capable of reducing the contact area with the clothes or the like, and can therefore realize smooth sleeve pass.

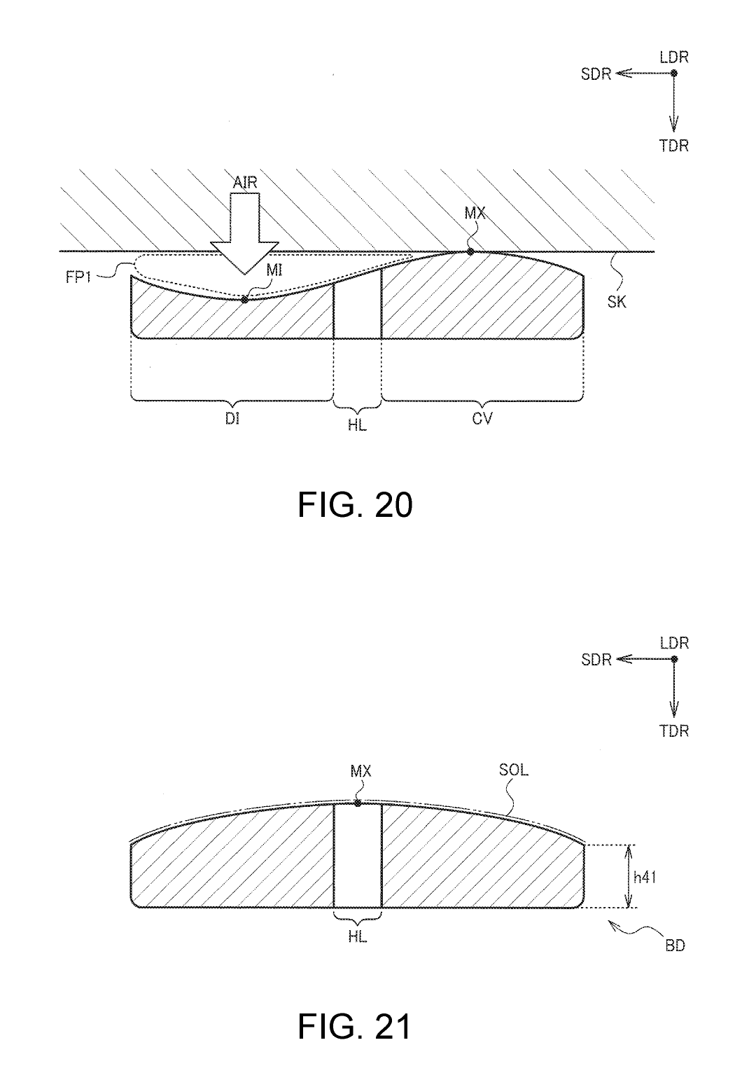

Thus, as shown in FIG. 20, when the test subject wears the band BD (or the physical activity information detection section), the region corresponding to the convex part CV of the reverse surface 21 abuts on (has contact with) the skin (the mounting region, the surface) SK of the test subject. In contrast, as indicated by FP1 shown in FIG. 20, since the band BD is lifted from (floats above) the skin SK of the test subject with the convex part CV as a pivot point, even in the case in which the test subject wears the band BD, a region not abutting on the skin of the test subject is created in the region other than the convex part CV of the reverse surface 21. Thus, it becomes possible to make the air (AIR in FIG. 20) enter the space between the region not abutting on the skin of the test subject and the skin on the test subject while mounting the band BD, and thus the airflow can be improved. Therefore, the discomfort caused while wearing the band can be prevented. For example, it becomes possible to prevent the itch, the pain, and the rash in the mounting region of the band 11 from occurring.

Further, as shown in FIG. 16, the contour line SOL on the reverse surface 21 side has a concave part DI. Further, in the case of defining the height from the reference surface BS to the concave part DI as h3, the height h3 from the reference surface BS to the concave part DI is lower than the height h1 from the reference surface BS to the hole position HLP. In other words, h2>h1>h3 is true, and thus the concave part DI is formed at a lower position than the hole position HLP.

Here, the concave part DI corresponds to a given range including a bottom part MI at which the distance from the reference surface BS becomes the shortest in the contour line SOL on the reverse surface side of the cross-section along the short-side direction SDR of the band BD. Further, the bottom part MI denotes a given range including a bottom point (bottom side) at which the wavy shape formed by the contour line SOL is switched from falling to rising. The bottom part MI can be said to be a local minimum point of the wavy shape. It should be noted that the bottom part MI can also be a flat bottom side. For example, although in the example shown in FIG. 16 and FIG. 17, the contour line SOL can be divided into two ranges with reference to the fitting hole HL (or the hole position HLP) , the concave part DI is the range including the local minimum point MI out of the two ranges.

Thus, as shown in FIG. 20, when the test subject wears the band BD, the region corresponding to the convex part CV of the reverse surface 21 and the skin of the test subject become in an abutting state (contact state), and the region corresponding to the concave part DI of the reverse surface 21 and the skin of the test subject become in a non-abutting state (non-contact state) . Therefore, it becomes possible to form a space (FP1 in FIG. 20) between the region corresponding to the concave part DI of the reverse surface 21 and the skin of the test subject when mounting the band BD, and it becomes possible, for example, to make the air (AIR in FIG. 20) flow through the space. It should be noted that in the present embodiment, it is not required for such a space between the band and the skin SK as FP1 shown in FIG. 20 to be always ensured, but it is possible to provide such small unevenness as to temporarily generate a space due to a motion of the arm of the user.

Further, as shown in FIG. 16, the band BD has a first side surface SS1 and a second side surface SS2 crossing the reverse surface and the obverse surface. Further, a position corresponding to the first side surface SS1 in the reverse surface is defined as a first side surface position SP1, a position corresponding to the second side surface SS2 in the reverse surface is defined as a second side surface position SP2, and the height from the reference surface BS to the side surface position SP1 is defined as h4. In this case, the height h4 from the reference surface BS to the side surface position SP1 is lower than the height h2 from the reference surface BS to the convex part CV. In other words, h2>h4 is true. It should be noted that in the example shown in FIG. 16, the height from the reference surface BS to the side surface position SP2 is also h4, and is the same as the height from the reference surface BS to the side surface position SP1.

Here, the side surface position SP1 (SP2) denotes the intersection between the contour line SOL on the reverse surface side and the side surface SS1 (SS2) in, for example, the cross-section along the short-side direction SDR of the band BD. In other words, the side surface position SP1 (SP2) is a position of an end (or the other end) on the side surface side of the band BD in the contour line SOL on the reverse surface side.

Incidentally, for example, when the test subject wears the band and the band is displaced laterally, or when the test subject wears the band for a long period of time, the contour line (e.g., SSOL in FIG. 3) on the reverse surface side of the side surface of the band is pressed against the skin of the test subject, and the test subject feels a pain or a trace of the band remains on the skin in some cases. This phenomenon is apt to occur in the case in which the contour line on the reverse surface side of the side surface of the band has a wavy shape.

In contrast, as shown in FIG. 16 and FIG. 17, in the band according to the present embodiment, the height h4 from the reference surface BS to the side surface position SP1 (SP2) becomes constant. In this case, as shown in FIG. 15 described above, the contour line SSOL on the reverse surface side of the side surface of the band has a non-wavy shape. For example, the contour line SSOL on the reverse surface side of the side surface of the band becomes a straight line.

Thus, it is possible to prevent the test subject from feeling the pain, or to prevent the trace of the band from remaining on the skin when the band is laterally displaced or when the test subject wears the band for a long period of time.

To wrap up, the relation of the height from the reference surface BS in the cross-section in the short-side direction of the band BD fulfills h2>h1>h3 and h2>h4>h3.

Thus, as shown in FIG. 16, it becomes possible, for example, to provide the region (the region corresponding to the convex part CV) protruding to a level higher than the hole position HLP and the region (the region corresponding to the concave part DI) recessed to a level lower than the hole position HLP to the reverse surface of the band BD in the cross-section in the short-side direction of the band BD. Further, as shown in FIG. 15, FIG. 16, FIG. 17 and so on, it becomes possible, for example, to make the height h4 of the side surface SS1 (SS2) of the band BD from the reference surface BS, for example, constant in the cross-section in the short-side direction of the band BD.

Further, in the G-G′ cross-section shown in FIG. 17, the positional relationship between the convex part CV and the concave part DI is reversed from that in the example of the F-F′ cross-section shown in FIG. 16, but the height relationship between the respective sections fulfills h2>h1>h3 similarly to the example shown in FIG. 16. Further, h2>h4>h3 is true.

In other words, in the present embodiment, in the cross-section in the short-side direction of the band BD, in the case of using the reference surface BS as a reference, the region corresponding to the convex part CV protrudes to the highest level, and the region corresponding to the concave part DI is recessed to the lowest level. Further, the hole position HLP and the side surface position SP1 (SP2) are positions located between the local maximum point MX of the convex part CV and the local minimum point MI of the concave part DI. It should be noted that in the example shown in FIG. 16 and FIG. 17, the magnitude relation between the height h1 from the reference surface BS to the hole position HLP and the height h4 from the reference surface BS to the side surface position SP1 (SP2) does not matter. In other words, either one of the hole position HLP and the side surface position SP1 (SP2) can be higher or lower than the other thereof with reference to the reference surface BS, or the hole position HLP and the side surface position SP1 (SP2) are the same in height.

Further, the band BD according to the present embodiment is formed of such cross-sectional shapes as shown in FIG. 16 and FIG. 17 folding alternately on one another. It should be noted that as shown in FIG. 6, FIG. 13, and FIG. 14, in the long-side direction LDR of the band BD, between the F-F′ cross-sectional shape shown in FIG. 16 and the G-G′ cross-sectional shape shown in FIG. 17, there exists a region MA for continuously and smoothly connecting the convex part CV shown in FIG. 16 to the concave part DI shown in FIG. 17, and continuously and smoothly connecting the concave part DI shown in FIG. 16 to the convex part CV shown in FIG. 17. Thus, as shown in FIG. 13 and FIG. 14 described above, the contour line OL of the reverse surface 21 has the wavy shape in the long-side direction LDR of the band.

As described hereinabove, the band 11 according to the present embodiment has the wavy shape in both of the short-side direction SDR of the reverse surface 21 and the long-side direction LDR thereof. As a result, as shown in FIG. 3, it becomes possible to make it easy for the air to enter the space between the mounting region of the band 11 and the band 11 to thereby improve the airflow, for example. Therefore, it becomes possible to prevent the itch, the pain, and the rash in the mounting region of the band 11 from occurring.

Further, as shown in FIG. 16 and FIG. 17, the fitting holes 16 are each formed at a position except the highest position of the convex part CV and the lowest position of the concave part DI. In other words, the fitting holes 16 are each formed at a position except the position (the local maximum point MX) where the distance from the reference surface BS becomes the longest and the position (the local minimum point MI) where the distance from the reference surface BS becomes the shortest.

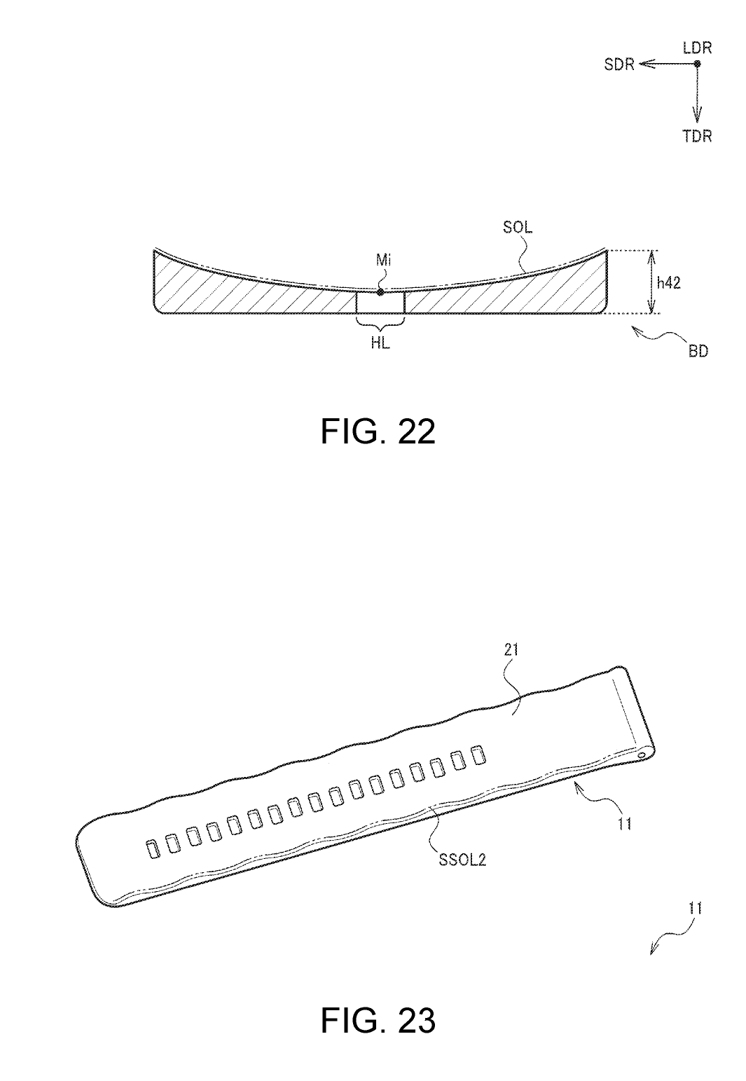

For example, FIG. 21 and FIG. 22 each show a comparative example of the band according to the present embodiment. In the example shown in FIG. 21, unlike the band according to the present embodiment, in the cross-section along the short-side direction SDR of the band BD, the fitting hole HL is formed in a region including the local maximum point MX of the contour line SOL of the reverse surface. Further, in the example shown in FIG. 22, in the cross-section along the short-side direction SDR of the band BD, the fitting hole HL is formed in a region including the local minimum point MI of the contour line SOL of the reverse surface.

Here, the height of the side surface of the band BD shown in FIG. 21 is defined as h41, and the height of the side surface of the band BD shown in FIG. 22 is defined as h42, and h42>h41 is assumed. Further, in the case in which the band BD is formed of such cross-sectional shapes as shown in, for example, FIG. 21 and FIG. 22 folding alternately on one another, the contour line SSOL2 of the side surface in the reverse surface of the band BD has the wavy shape as shown in FIG. 23. Therefore, as described above, for example, when the test subject wears the band and the band is displaced laterally, or when the test subject wears the band for a long period of time, the contour line (e.g., SSOL in FIG. 3) on the reverse surface side of the side surface of the band is pressed against the skin of the test subject, and the test subject feels a pain or a trace of the band remains on the skin in some cases. Further, if the fitting hole HL is formed in the region including the local maximum point MX in the cross-section along the short-side direction SDR of the band BD as shown in FIG. 21, the edge of the fitting hole HL abuts on the mounting region of the test subject while mounting the band to cause the test subject to feel the pain, or to provide a trace of the fitting hole HL to the mounting region in some cases.

In contrast, in the band according to the present embodiment, the fitting hole 16 is formed at a position except the local maximum point MX and the local minimum point MI in the cross-section along the short-side direction SDR of the band 11 as described above. Therefore, as shown in FIG. 15, FIG. 16, FIG. 17 and so on described above, the height of the side surface of the band BD can be made constant. If the contour line on the reverse surface side of the band BD is a straight line without unevenness, even in the case in which, for example, the contour line on the reverse surface side of the band BD abuts on the skin of the test subject, it becomes possible, for example, to prevent the pain or the band trace while mounting the band from occurring. Further, as described above, it becomes possible, for example, to prevent the edge of the fitting hole HL or the contour line of the side surface of the band from abutting on the mounting region of the test subject while mounting the band BD to cause the test subject to feel the pain, or to provide a trace of the fitting hole HL to the mounting region.

Further, since the fitting holes 16 are formed at the positions described above, the contour line of the fitting hole 16 on the reverse surface 21 side in the cross-section along the short-side direction of the band 11 is tilted with respect to the reference surface BS as indicated by the arrow YS shown in FIG. 16 and FIG. 17.

Thus, for example, it becomes possible to make the height h5 from the reference surface BS to one end HP1 of the fitting hole 16 and the height h6 from the reference surface BS to the other end HP2 of the fitting hole 16 different from each other as shown in FIG. 16 and FIG. 17. Further, in other words, the height from the reference surface BS can be made different between the region on the first direction side with respect to the hole position HLP and the region on the second direction side opposite to the first direction with respect to the hole position HLP. It should be noted that in the example shown in FIG. 16, the region on the first direction side with respect to the hole position HLP is the region corresponding to the convex part CV, and the region on the second direction side with respect to the hole position HLP is the region corresponding to the concave part DI. As a result, as described above, it becomes possible, for example, to improve the airflow of the band.

Further, as shown in FIG. 16 and FIG. 17, it can be said that the contour line SOL on the reverse surface 21 side in the cross-section along the short-side direction of the band 11 is point symmetrical about the hole position HLP. It should be noted that in the present embodiment, the contour line SOL is not limited to be strictly point symmetrical about the hole position HLP, but it is sufficient for the contour line SOL to be substantially point symmetrical. Further, in the case of setting the hole position HLP to the center of the contour line SOL, it can be said that the fitting hole HL is set so as to include the hole position HLP.

Thus, it becomes possible, for example, to set the shape of the contour line SOL on the reverse surface of the band in the cross-section in the short-side direction SDR of the band to the reversed shape cantered on the hole position HLP. As a result, as described above, it becomes possible, for example, to form the region protruding with respect to the reference surface and the region recessed with respect to the reference surface in the reverse surface of the band to arrange that the air enters the space while mounting the band.

Further, focusing attention on the thickness of the band, in the present embodiment, it is desirable for the thickness (h2−h1) between the convex part CV and the hole position HLP to be no smaller than 0.10 mm and no larger than 1.00 mm. Further, regarding the thickness (h3−h2) between the convex part CV and the concave pert DI, it is desirable to be no smaller than 0.25 mm and no larger than 1.50 mm.

Thus, it becomes possible, for example, to form an entrance path of the air between the band and the skin of the test subject while ensuring the thinness of the band BD.

Therefore, it becomes possible, for example, to prevent the itch, the pain, and the rash from occurring in the mounting region of the band 11 while keeping the easiness in mounting the band and wearing feeling in good condition.

### 4. MODIFIED EXAMPLE

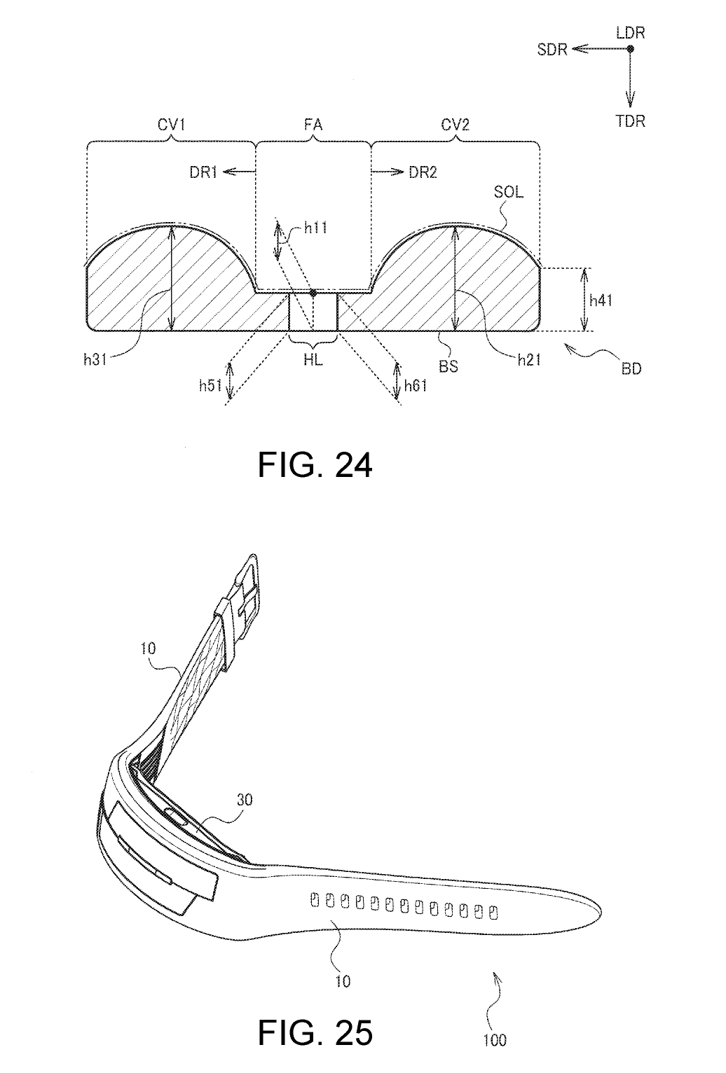

Then, a modified example of the present embodiment will be described using FIG. 24. In the present modified example, as shown in FIG. 24, the contour line SOL on the reverse surface side in the cross-section along the short-side direction SDR of the band BD has a convex part CV1 (CV2) and a flat part FA, and the flat part FA is provided with the fitting hole HL.

Specifically, in the example shown in FIG. 24, in the reverse surface in the cross-section along the short-side direction SDR of the band BD, the contour line SOL forms the convex part CV1 on the first direction DRi side with respect to the flat part FA, and the contour line SOL forms the convex part CV2 on the second direction DR2 side as an opposite direction to the first direction DRi with respect to the flat part FA. Further, the flat part FA located between the convex part CV1 and the convex part CV2 is provided with the fitting hole HL. The height relationship from the reference surface BS in the cross-section in the short-side direction SDR of the band BD in this case fulfills h21≧M31>h11. Further, h11=h51=h61 is true.

According also to such a configuration, it becomes possible, for example, to make it easy for the air to enter the flat part FA located between the convex part CV1 and the convex part CV2. As a result, as described above, it becomes possible, for example, to improve the airflow when mounting the band while keeping the comfort when mounting the band.

Further, by optimizing the wave number, the wavelength, the amplitude, and so on of the wavy shape, the airflow can further be improved.

For example, the wave number of the wavy shape on the reverse surface side in the cross-section along the short-side direction of the band 11 is desirably equal to or smaller than 2.

As described above, the wavy shape is formed of, for example, a certain aperiodic wave repeated periodically. In this case, the wave number of the wavy shape denotes the number of waves included in the wavy shape. Specifically, the wave number of the wavy shape on the reverse surface 21 side in the cross-section along the short-side direction of the band 11 is more preferably equal to 1. For example, in FIG. 16 described above, the wave number of the wavy shape on the reverse surface 21 side in the F-F′ cross-section shown in FIG. 6 is set to 1. In this case, the wavy shape itself can be said to be an aperiodic wave.

Further, the wavelength of the wavy shape in the reverse surface 21 of the band section is set to be longer than the length in the short-side direction of the obverse surface 22 of the band section. It should be noted that the wavelength of the wavy shape denotes the length corresponding to the twice of the distance between the peak part and the bottom part in the wavy shape toward the short-side direction of the band. For example, the wavelength of the wavy shape denotes the length twice as long as the distance in the short-side direction SDR from the local maximum point (the peak part) MX to the local minimum point (the bottom part) MI. Further, the length in the short-side direction of the obverse surface 22 denotes the distance between the first side surface SS1 and the second side surface SS2 in the example shown in FIG. 16, for example.

Further, the amplitude of the wavy shape denotes the distance in the thickness direction of the band 11 between the peak part and the bottom part of the wavy shape. For example, the amplitude of the wavy shape denotes the distance in the thickness direction TDR between the local maximum point (the peak part) MX to the local minimum point (the bottom part) MI in the example shown in FIG. 16.

Thus, it becomes possible, for example, to further improve the airflow.

Further, as shown in FIG. 6 described above, the first band 11 can also be provided with the wavy shape in at least the forming area 17 of the plurality of fitting holes 16.

Here, the forming area 17 of the plurality of fitting holes 16 denotes the region from the position of the fitting hole the closest to one end of the first band 11 out of the plurality of fitting holes 16 to the position of the fitting hole the closest to the other end of the first band 11 out of the plurality of fitting holes 16. In the first band 11, the forming area 17 of the plurality of fitting holes 16 is the easiest to have contact with the skin of the test subject.

Thus, it becomes possible, for example, to provide the wavy shape to the part having contact with the skin of the test subject out of the first band 11.

Further, as shown in FIG. 1 and FIG. 2 described above, it is also possible for the second band 12 to be provided with the wavy shape from the buckle section (or the buckle) 14 to the connection region with the case section 30.

Thus, it becomes possible, for example, to provide the wavy shape to the part having contact with the skin of the test subject out of the second band 12.

As described above, according to the present embodiment, only by changing the shape of the band, it is possible to obtain the suppression effect of the itch, the pain, the rash, and so on while mounting the band without adding a special material or component.

Besides the above, in the present embodiment it is also possible to perform the following process on the band to increase the variety of effects.

For example, by using a material (e.g., silicone) high in heat radiation property as the material of the band, it is possible to enhance the effect of reducing the stuffy state or wetting due to sweating. Further, by using a material (e.g., silicone) weak in stimulus to the skin as the material of the band, the itch or the rash can be reduced.

Further, by applying a material (e.g., a fluorine material) for decreasing the frictional resistance value on the surface of the band, it is possible to reduce the stickiness of the surface of the band to reduce the itch and the rash.

Further, it is also possible to provide microscopic unevenness to the surface of the band by applying embossing on the surface of the wavy shape of the band. Here, embossing is a kind of surface treatment, and denotes a process of physically providing a wrinkle pattern (the microscopic unevenness) . Thus, it is possible to increase the air contact, and it becomes possible, for example, to reduce the stuffy state and wetting.

Alternatively, it is also possible to provide microscopic waves to the surface of the wavy shape of the band. For example, a fractal shape can also be provided. Thus, it is possible to further reduce the stuffy state and wetting.

Further, by providing the wavy shape not only to the band but also to the reverse side (the wrist side) of the case section, an increase in the effect can be expected.

Further, the present embodiment described hereinabove can be applied not only to the portable electronic apparatus 100 having the first band 11 and the second band 12 as shown in FIG. 1, but also to the portable electronic apparatus 100 having a single band 10 alone as shown in FIG. 25. In the portable electronic apparatus 100 shown in FIG. 25, the case section 30 is disposed on the reverse surface of the band 10.

Although the present embodiment is hereinabove described in detail, it should easily be understood by those skilled in the art that it is possible to make a variety of modifications not substantially departing from the novel matters and the advantages of the invention. Therefore, all of such modified examples should be included in the scope of the invention. For example, a term described at least once with a different term having a broader sense or the same meaning in the specification or the accompanying drawings can be replaced with that different term in any part of the specification or the accompanying drawings. Further, the configurations and the operations of the portable electronic apparatus are not limited to those described in the present embodiment, but can be put into practice in variously modified forms.

What is claimed is:

1. A portable electronic apparatus comprising:
a band adapted to be mounted on a test subject,
wherein the band includes
a plurality of fitting holes,
a first surface as a surface facing to the test subject when mounted, and
a second surface as a surface facing to an opposite side to the test subject when mounted,
a contour line on the first surface side in a cross-section along a short-side direction of the band has a convex part, and
in a case of defining the second surface as a reference surface, and a position corresponding to the fitting hole in the first surface as a hole position, a height from the reference surface to the hole position is lower than a height from the reference surface to the convex part.

2. The portable electronic apparatus according to claim 1, wherein
the contour line on the first surface side in the cross-section along the short-side direction of the band has the convex part and a concave part.

3. The portable electronic apparatus according to claim 2, wherein
in a case of defining the height from the reference surface to the hole position as h1, the height from the reference surface to the convex part as h2, and a height from the reference surface to the concave part as h3, h2>h1>h3 is true.

4. The portable electronic apparatus according to claim 2, wherein
the band has a first side surface and a second side surface each crossing the first surface and the second surface, and
in a case of defining a position corresponding to the first side surface and the second side surface in the first surface as a side surface position, a height from the reference surface to the convex part as h2, and a height from the reference surface to the side surface position as h4, h2>h4 is true.

5. The portable electronic apparatus according to claim 4, wherein
in a case of defining a height from the reference surface to the concave part as h3, h2>h4>h3 is true.

6. The portable electronic apparatus according to claim 3, wherein
0.10 mm≦(h2−h1)≦1.0 mm is true.

7. The portable electronic apparatus according to claim 1, wherein
the fitting hole is formed at a position except the highest position of the convex part.

8. The portable electronic apparatus according to claim 1, wherein
the contour line on the first surface side in the cross-section along the short-side direction of the band has the convex part and a concave part, and
the fitting hole is formed at a position except the highest position of the convex part and the lowest position of the concave part.

9. The portable electronic apparatus according to claim 1, wherein
a contour line of the fitting hole on the first surface side in the cross-section along the short-side direction of the band is tilted with respect to the reference surface.

10. The portable electronic apparatus according to claim 1, wherein
the contour line on the first surface side in the cross-section along the short-side direction of the band is point symmetrical about a central part of the hole position.

11. The portable electronic apparatus according to claim 1, wherein
in the band, a contour line on the first surface side in a cross-section along a long-side direction of the band has a wavy shape.

12. The portable electronic apparatus according to claim 1, wherein
the contour line on the first surface side in the cross-section along the short-side direction of the band has a convex part and a flat part, and the fitting hole is disposed in the flat part.

13. The portable electronic apparatus according to claim 1, further comprising:
a physical activity information detection section adapted to detect physical activity information of the test subject.

14. A portable electronic apparatus comprising:
a band adapted to be mounted on a test subject,
wherein the band includes
a plurality of fitting holes,
a first surface as a surface facing to the test subject when mounted, and
a second surface as a surface facing to an opposite side to the test subject when mounted,
a contour line on the first surface side in a cross-section along a short-side direction of the band has a convex part, and
in a case of defining the second surface as a reference surface, the fitting hole is formed at a position except a position where a distance from the reference surface becomes the longest in the cross-section along the short-side direction of the band.

15. The portable electronic apparatus according to claim 14, wherein
the contour line on the first surface side in the cross-section along the short-side direction of the band has the convex part and a concave part.

16. The portable electronic apparatus according to claim 14, wherein
the contour line on the first surface side in the cross-section along the short-side direction of the band has the convex part and a concave part, and
the fitting hole is formed at a position except the highest position of the convex part and the lowest position of the concave part.

17. The portable electronic apparatus according to claim 14, wherein
the contour line on the first surface side in the cross-section along the short-side direction of the band is point symmetrical about a central part of the hole position.

18. The portable electronic apparatus according to claim 14, wherein
in the band, a contour line on the first surface side in a cross-section along a long-side direction of the band has a wavy shape.

19. The portable electronic apparatus according to claim 14, wherein
the contour line on the first surface side in the cross-section along the short-side direction of the band has a convex part and a flat part, and the fitting hole is disposed in the flat part.

20. The portable electronic apparatus according to claim 14, further comprising:
a physical activity information detection section adapted to detect physical activity information of the test subject.
